(19) United States
(12) Patent Application Publication
(10) Pub. No.: US 2011/0300044 A1
(43) Pub. Date: Dec. 8, 2011

(54) CORROSION INHIBITORS

(75) Inventors: Paitoon Tontiwachwuthikul, Regina (CA); Raphael Idem, Reginia (CA); Nattawan Kladkaew, Phetchaburi (TH); Chintana Saiwan, Vudtakad (TH)

(73) Assignee: University of Regina, Regina (CA)

(21) Appl. No.: 13/101,945

(22) Filed: May 5, 2011

Related U.S. Application Data
(60) Provisional application No. 61/331,730, filed on May 5, 2010.

(51) Int. Cl. B01D 53/40; C09K 3/00; C07D 295/027; B01D 53/62; C07C 211/07; C01G 39/00

(52) U.S. Cl. 423/228; 564/463; 423/594.15; 544/106; 423/210; 423/220; 252/184

(57) ABSTRACT

Corrosion inhibitors and processes, uses, methods, compositions, devices, and apparatus involving corrosion inhibitors are disclosed. In certain embodiments the use of specific corrosion inhibitors, specific concentrations of inhibitors, blends of inhibitors, processes utilizing such inhibitors, and compositions comprising such inhibitors, for example in acid gas separation systems such as aqueous amine- or ammonia-based CO₂ separation systems (e.g., the post combustion capture of CO₂ from flue gases using amines). According to some embodiments, corrosion inhibitors are used in a process for separating at least a portion of an acid gas from a gaseous mixture wherein the inhibitor is at least one selected from dodecylamine, sodium molybdate, morpholine, or a combination thereof.

This application is based on and claims domestic priority benefits under 35 USC §119(e) from U.S. Provisional Application Ser. No. 61/331,730 filed May 5, 2010, the entire content of which is expressly incorporated hereinto by reference.

This disclosure relates to the use of corrosion inhibitors techniques in a solvent-based post combustion capture system.

Combustion flue gases, refinery off gas, and reformate gas, and the production and use of fossil fuels contribute to an increase in emissions of greenhouse gases (GHGs), such as acid gases—especially carbon dioxide (CO₂) and other pollutants such as oxides of sulfur (SOₓ), oxides of nitrogen (NOₓ), hydrogen sulphide (H₂S) and hydrogen chloride (HCl). It is desirable to reduce the emissions of CO₂ or the other pollutants. As a result, large sources of CO₂ emissions such as coal-fired power plants, refineries, cement manufacturing, and the like have been targeted to attempt to reduce and/or recover the CO₂ emitted.

One method of acid gas capture is gas absorption using aqueous amine solutions or ammonia solutions. Typically this method is used to absorb CO₂ and H₂S from low-pressure streams such as flue gases emitted from power plants. An example of an amine used in this type of process is monoethanolamine, MEA.

In a typical system, CO₂ capture by absorption using chemical liquid absorbent involves absorbing CO₂ from the flue gas stream into the absorbent flowing down from the top of the absorber column counter-currently with the flue gas stream, which flows upwards from the bottom of the column. The CO₂ rich liquid from the absorber column is then pumped through the lean/rich exchanger to the top of the stripper column where CO₂ is stripped off the liquid by application of steam through a reboiler thereby regenerating the liquid absorbent. The chemical absorption of CO₂ into the liquid absorbent in the absorber is exothermic leading to the release of heat. The stripping of CO₂ from the liquid absorbent in the stripper is endothermic and requires external heating. Typically the lowest temperature in the absorber column is no higher than 60° C., which is limited by the temperatures of the lean liquid absorbent and flue gas stream temperatures, and the highest temperature is around 90° C. The typical temperature for stripping or desorption is in the range of 105-150° C. The CO₂ desorption process is endothermic with a much higher heat demand than the absorption process can provide thus setting up a temperature mismatch between the absorber and regenerator/stripper.

Aqueous amine-based CO₂ capture systems can suffer from significant corrosion problems. Corrosion can affect almost every part of the process equipment depending on operating parameters such as amine type, amine concentration, CO₂ loading, process temperature, oxygen concentration, presence of degradation products, and solution velocity. Attempts have been made to minimize corrosion through the design and operation of the plant, the use of corrosion-resistant materials, the removal of corrosion-promoting agents, and the use of corrosion inhibitors.

The use of a corrosion inhibitor can be economical and does not generally require major process modifications for existing plants. Various corrosion inhibitors have been developed and commercialized for use in amine treating units. Inorganic inhibitors have been favoured over organic compounds because of their superior inhibition performance. However, these inorganic corrosion inhibitors (e.g. inhibitors containing toxic arsenic, antimony and vanadium) are not considered environmentally friendly. Vanadium compounds, particularly sodium metavanadate, are used extensively in amine treating plants. This compound can be toxic and is also detrimental to the rate of degradation of the amine (e.g. MEA).

The present disclosure relates to corrosion inhibitors and processes, uses, methods, compositions, devices, and apparatus involving corrosion inhibitors. For example, the present disclosure provides the use of specific corrosion inhibitors, specific concentrations of inhibitors, blends of inhibitors, processes utilizing such inhibitors, and compositions comprising such inhibitors.

The present disclosure provides the use of specific corrosion inhibitors in acid gas separation systems such as aqueous amine- or ammonia-based CO₂ separation systems. An example of the mentioned separation processes is the post combustion capture of CO₂ from flue gases using amines.

The present technology may be used in a variety of situations. For example, in the treatment of:
Exhaust gases from electric power generating plants
Exhaust and off gases from breweries and ethanol plants
Exhaust and off gases from cement manufacturing plants
Refinery off gases
Reformate gas or product gas mixture from reforming plants to produce hydrogen
Biogas
Combustion flue gas to produce steam for steam assisted gravity drainage (SAGD) operations for crude oil and oil sands production
Natural gas processing

The present disclosure may be applied to amine-based or ammonia-based methods for CO₂ absorption and/or desorption. This includes using different types of amines and/or absorbents, different process configurations, and using steam and/or hot water to provide the energy that is required for stripping for CO₂ capture from flue gas streams, natural gas, reformate gas, etc.

### BRIEF DESCRIPTION OF THE DRAWINGS

FIG. 1 schematically depicts an experimental setup to study corrosion and corrosion behaviour of carbon steel C1020 in MEA-H₂O—CO₂—O₂—SO₂ system;

FIG. 2 are polarization curves using imidazole as a corrosion inhibitor at different concentrations;

FIG. 3 is a plot of corrosion rate (mpy) versus imidazole concentration (ppm);

FIG. 4 is plot of percent inhibition efficiency versus imidazole concentration (ppm);

FIG. 5 are polarization curves using dodecylamine as a corrosion inhibitor at different concentrations;

FIG. 6 is a plot of corrosion rate (mpy) versus dodecylamine concentration (ppm);

FIG. 7 is plot of percent inhibition efficiency versus dodecylamine concentration (ppm);

FIG. 8 are polarization curves using sodium molybdate as a corrosion inhibitor at different concentrations;

FIG. 9 is a plot of corrosion rate (mpy) versus sodium molybdate concentration (ppm);

FIG. 10 is plot of percent inhibition efficiency versus sodium molybdate concentration (ppm);

FIG. 11 are polarization curves using morpholine as a corrosion inhibitor at different concentrations;

FIG. 12 is a plot of corrosion rate (mpy) versus morpholine concentration (ppm);

FIG. 13 is plot of percent inhibition efficiency versus morpholine concentration (ppm);

FIG. 14 are polarization curves using a commercial corrosion inhibitor at different concentrations;

FIG. 15 is a plot of corrosion rate (mpy) versus commercial corrosion inhibitor concentration (ppm);

FIG. 16 is plot of percent inhibition efficiency versus commercial corrosion inhibitor concentration (ppm);

FIG. 17 are polarization curves using imidazole, morpholine, and imidazole/morpholine as corrosion inhibitors;

FIG. 18 is a plot of corrosion rate (mpy) versus corrosion inhibitor concentration (ppm) for each of imidazole, morpholine, and imidazole/morpholine;

FIG. 19 is plot of percent inhibition efficiency versus corrosion inhibitor concentration (ppm) for each of imidazole, morpholine, and imidazole/morpholine;

FIG. 20 are polarization curves using dodecylamine, morpholine and dodecylamine/morpholine as corrosion inhibitors;

FIG. 21 is a plot of corrosion rate (mpy) versus corrosion inhibitor concentration (ppm) for each of dodecylamine, morpholine and dodecylamine/morpholine;

FIG. 22 is plot of percent inhibition efficiency versus corrosion inhibitor concentration (ppm) for each of dodecylamine, morpholine and dodecylamine/morpholine;

FIG. 23 are polarization curves using sodium molybdate, morpholine, and sodium molybdate/morpholine as corrosion inhibitors;

FIG. 24 is a plot of corrosion rate (mpy) versus corrosion inhibitor concentration (ppm) for each of sodium molybdate, morpholine, and sodium molybdate/morpholine; and

FIG. 25 is plot of percent inhibition efficiency versus corrosion inhibitor concentration (ppm) for each of sodium molybdate, morpholine, and sodium molybdate/morpholine.

In the description that follows, a number of terms are used, the following definitions are provided to facilitate understanding of various aspects of the disclosure. Use of examples in the specification, including examples of terms, is for illustrative purposes only and is not intended to limit the scope and meaning of the embodiments of the invention herein. Numeric ranges are inclusive of the numbers defining the range. In the specification, the word “comprising” is used as an open-ended term, substantially equivalent to the phrase “including, but not limited to,” and the word “comprises” has a corresponding meaning.

All citations are herein incorporated by reference, as if each individual publication was specifically and individually indicated to be incorporated by reference herein and as though it were fully set forth herein. Citation of references herein is not to be construed nor considered as an admission that such references are prior art to the present invention.

One or more currently preferred embodiments of the disclosure have been described by way of example. The invention includes all embodiments, modifications and variations substantially as hereinbefore described and with reference to the examples and figures. It will be apparent to persons skilled in the art that a number of variations and modifications can be made without departing from the scope of the invention as defined in the claims. Examples of such modifications include the substitution of known equivalents for any aspect of the invention in order to achieve the same result in substantially the same way.

As used herein, the term ‘absorption media’ and ‘adsorption media’ refers to media that can absorb/adsorb an amount of acid gas.

As used herein, the term ‘rich absorption and/or adsorption media’ refers to media that has absorbed/adsorbed an amount of acid gas relative to lean media.

As used herein, the term ‘lean absorption and/or adsorption media’ refers to media that has no or low amounts of acid gas.

Absorption/adsorption media that may be used herein include monoethanolamine (MEA), diglycolamine (DGA), diethanolamine (DEA), methyldiethanolamine (MDEA), 2-amino-2-methyl-1-propanol (AMP), piperazine (PZ), ammonia, amines, alkanolamines, amino alcohols, diamines, ionic liquids, aminosilicone, derivatives and/or combinations thereof.

As used herein, the term ‘acid gas’ refers to gases that form acidic solutions when mixed with water. Examples of acid gases include carbon dioxide (CO₂), sulphur dioxide (SO₂), sulphur trioxide (SO₃), hydrogen sulphide (H₂S), hydrogen chloride (HCl), and oxides of nitrogen (NOₓ).

It has been reported that the corrosion rate of carbon steel in MEA-H₂O—CO₂—O₂—SO₂ system increases with increasing MEA concentration, CO₂ loading, operating temperature, O₂ and SO₂ concentrations in the flue gas stream. Several corrosion mechanisms in MEA-H₂O—CO₂—O₂—SO₂ system have been postulated. The general anodic reaction in anode site is the dissolution of iron or the oxidation of iron to the ferrous (Fe²⁺) ion

Fe [symbol] Fe²⁺+2e⁻ (1)

There are different types of the reduction reactions (cathodic partial reactions) in MEA-H₂O—CO₂—O₂—SO₂ system on the cathode site.
Reduction of dissolved oxygen:

O₂+2H₂O+4e⁻ [symbol] 4OH⁻ (2)

Reduction of hydrogen ion (H⁺) or H₃O⁺

2H⁺+2e⁻ [symbol] H₂ or (3)

2H₃O⁺+2e⁻ [symbol] 2H₂O+H₂ (4)

Reduction of carbonic acid (H₂CO₃) and bicarbonate (HCO₃⁻)

2H₂CO₃+2e⁻ [symbol] H₂+2HCO₃⁻ (5)

2HCO₃⁻+2e⁻ [symbol] H₂+2CO₃²⁻ (6)

In our study on the efficacy and effect of concentration of various corrosion inhibitors was examined. The experiments were conducted under severe corrosion conditions found in the CO₂ capture process in which MEA, O₂, SO₂ concentrations, and CO₂ loading were 7 kmol/m³, 6%, 204 ppm, and 0.5 mol CO₂/mol MEA, respectively. The operating temperature of the corrosion experiments was at 353K. This condition represented an uninhibited system. MEA was chosen as the absorption solvent because of its widespread use in CO₂ capture process and its corrosiveness. Carbon steel¹² was used as a test specimen since it is widely used for construction of equipment in the CO₂ capture process. The objective of this work was to screen and test corrosion inhibitors and blends of corrosion inhibitors, including, dodecylamine, sodium molybdate, morpholine, imidazole, and a commercial inhibitor (MAX-AMINE CMX9053 from GE Betz).

### 2. Experiments—Equipment and Chemicals

The electrochemical experiments were carried out using monoethanolamine (reagent grade with >99% purity, Fisher Scientific, ON) as the absorption solvent. It was diluted with deionized water to the desired concentration, which was accurately determined by titration with 1.0 N hydrochloric acid (HCl) solution using methyl orange as a titration indicator. The desired concentration of aqueous MEA solution was then preloaded with carbon dioxide to obtain the desired CO₂ loading (mol CO₂/mol MEA) by purging a stream of CO₂ gas (Praxair, research grade, ON) into the solution. The CO₂ loading procedure followed the AOAC method¹³. The desired CO₂ loading was determined by titrating with the 1.0N HCl solution using a Chittick apparatus.

Carbon steel C1020 (Metal Samples Company, AL) was used to study the corrosion in MEA-H₂O—CO₂—O₂—SO₂ system. Its chemical composition in percentage is as follows: C, 0.19; Cr, 0.01; Cu, 0.01; Mn, 0.56; Mo, 0.01; N, 0.0036; Ni, 0.01; P, 0.009; S, 0.007; and Fe, balance. The tested specimens are cylindrical in shape with ⅜″ diameter, ½″ length with a 3-48 threaded hole at one end. The specimens were prepared in accordance with ASTM G1-90¹⁴. The specimens were wet ground with 240 grit silicon carbide paper, wet polished with 600-grit silicon carbide paper, rinsed with deionized water, dried with air and kept in a desiccator before use. The surface areas of the specimens were determined by measuring all dimensions with a vernier caliper.

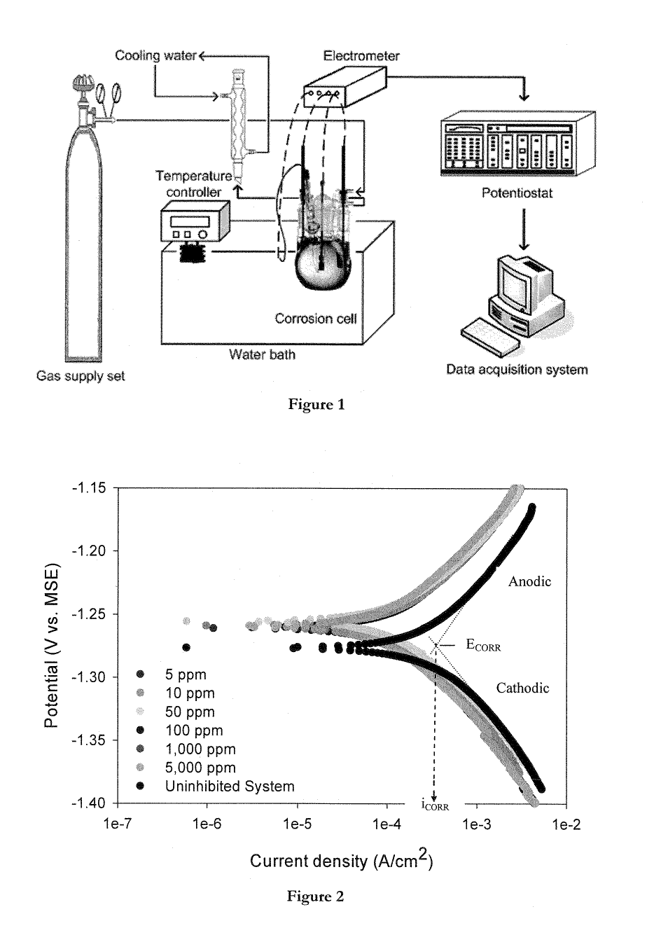

Electrochemical techniques were used to study corrosion and corrosion behaviour of carbon steel C1020 in MEA-H₂O—CO₂—O₂—SO₂ system. The experiment setup is shown in FIG. 1. It consists of an ASTM corrosion cell, potentiostat, water bath with temperature controller, condenser, gas supply set and data acquisition system. An ASTM corrosion cell model K47 (London Scientific, Ltd., ON), is a 1 liter flat bottom flask with ground glass joints. It is composed of one working electrode mounted with specimen; two high density carbon graphite rods use as counter electrode, one reference electrode which is a mercurous sulfate electrode (MSE), one bridge tube, a glass inlet and outlet for transferring gas to and from the corrosion cell. A potentiostat model 273A (London Scientific, Ltd., ON) was used to control the potential and to read the current accurately. PowerCORR version 2.47 (London Scientific, Ltd., ON) was used to acquire and analyse the experimental data. A water bath with a temperature controller was used to control the operating temperature. A condenser was connected to the corrosion cell to maintain the temperature in order to maintain the solution concentration in the cell by preventing evaporation during the experiment. The gas supply set was gaseous mixtures of SO₂—O₂—N₂ that were similar to actual flue gas stream conditions.

The ASTM G5-94¹⁵ was used in evaluating the accuracy of a given electrochemical test apparatus. It was performed by running the experiment with potentiodynamic anodic polarization technique on a stainless steel 430 in 1N sulfuric acid (H₂SO₄) solution at 30° C. The reliability of experiment is ascertained when the obtained polarization plot appears within the reference band. All of the electrochemical experiments were carried out in accordance with ASTM G5-94.

### 2.2 Corrosion Measurement Techniques

The Tafel plot technique^{16,17} was used to evaluate corrosion rate. A Tafel Plot was generated by beginning the scan from −250 mV to +250 mV vs. corrosion potential (E_{CORR}). The resulting data is plotted as the applied potential vs. the logarithm of the measured current. The corrosion current (i_{CORR}) was obtained from the intersection at E_{CORR}, and then was used to calculate the corrosion rate using equation 7.

$$CR ( mpy ) = \frac{0.13 {i}_{CORR} ( E . W . )}{A \times d} \qquad ( eq . 7 )$$

where CR is the corrosion rate in mpy (mils per year), i_{CORR} is the corrosion current in microampere (μA), E.W. is equivalent weight of the corroding species in gram (g), A is the surface area of the specimen in square centimeter (cm²) and d is the density of the specimen in gram per cubic centimeter (g/cm³).

### 2.3 Inhibition Efficiency

The inhibition efficiency of corrosion inhibitor was investigated in the system of severe corrosiveness, which was 7 kmol/m³ MEA, CO₂ loading of 0.5 mol CO₂/mol MEA with simulated flue gas stream of 204 ppm SO₂ and 6% O₂ at 353K. Its performance was calculated by the following equation¹⁸:

$$Inhibitor efficiency ( \% ) = 100 \frac{( {CR}_{uninhibited} - {CR}_{inhibited} )}{{CR}_{uninhibited}} \qquad ( eq . 8 )$$

where CR_{uninhibited} is corrosion rate of the uninhibited system and CR_{inhibited} is corrosion rate of the inhibited system.
2.4 Corrosion System without Inhibitor

The corrosion cell containing about 1 liter of 7 kmol/m³ MEA, CO₂ loading of 0.5 mol CO₂/mol MEA was immersed in a water bath with a temperature controller. The temperature of the solution was kept constant at 353 K. A stream of simulated flue gas of 204 ppm SO₂ and 6% O₂ was introduced into the corrosion cell at the flow rate of 150 ml/min for one and a half hour. The carbon rods counter electrodes were placed in the test cell. Then, the salt bridge was filled with a test solution and placed in the corrosion cell. The prepared surface was mounted on the electrode holder rod. Consequently, the specimen was degreased with methanol and rinsed in distilled water just prior to immersion in the test cell. The salt-bridge probe tip was adjusted close to the specimen electrode. All the lines between the corrosion cell and the Model 273A potentiostat had been connected before the corrosion potential (E_{CORR}) versus the MSE reference electrode of the test system were measured for at least 1 hour to ensure that the corrosion potential value remained constant. Finally, the electrochemical experiment was started. The applied potential and the measured current were continuously recorded.

2.4.2 Corrosion System with Inhibitor.

Various corrosion inhibitors were investigated their inhibition in amine based solvents for CO₂ absorption from power plant flue gases containing CO₂, O₂, and SO₂. They were dodecylamine, morpholine, and sodium molybdate. Imidazole and a commercially available inhibitor were also examined.

A known concentration of inhibitor was introduced into the corrosion cell containing 1 liter of 7 kmol/m³ MEA, CO₂ loading of 0.5 mol CO₂/mol MEA solution and the procedure repeated that described for the corrosion system without inhibitor.

2.4.3 Corrosion System with Blended Inhibitors Imidazole/Morpholine, Dodecylamine/Morpholine and Sodium Molybdate/Morpholine.

Dodecylamine, sodium molybdate, and imidazole were blended with morpholine to see if the combination produced better inhibition.

The optimum concentrations of single inhibitors were used in blending and testing for its performance. The blended inhibitors were introduced into the corrosion cell containing 1 liter of 7 kmol/m³ MEA, CO₂ loading of 0.5 mol CO₂/mol MEA solution and the procedure was repeated.

### 3. Results and Discussion

A realistic system consisting of 7 kmol/m³ MEA, CO₂ loading of 0.5 mol CO₂/mol MEA solution with simulated flue gas stream of 204 ppm SO₂ and 6% O₂ at 353 K was used through out all experiments to determine the optimum concentration of the tested inhibitor. This condition is called an uninhibited system gave the corrosion rate of carbon steel about 211 mpy.

3.1 Corrosion System with Inhibitor

3.1.1 Imidazole

The experiments were performed by adding imidazole in concentrations from 0 to 5,000 ppm. FIG. 2 shows the obtained Tafel plots of imidazole at different imidazole concentrations. Tafel plot technique produces anodic and cathodic polarization curves. The corrosion current i_{CORR} is obtained by the intersection of extrapolating the linear portion of the curve to E_{CORR}. The i_{CORR} can be used to calculate corrosion rate by using equation (7). Generally, a higher i_{CORR} shows a higher corrosion rate. The addition of imidazole into the system shows a lower i_{CORR}, since the polarization curve of the inhibited system is shifted to a lower current density, indicating a lower corrosion rate.

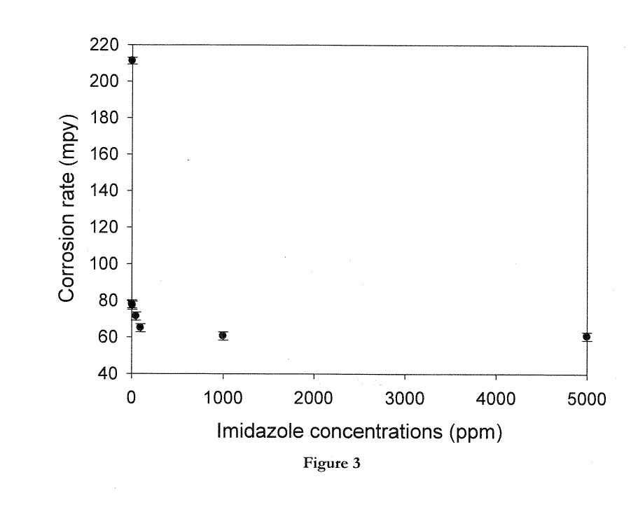

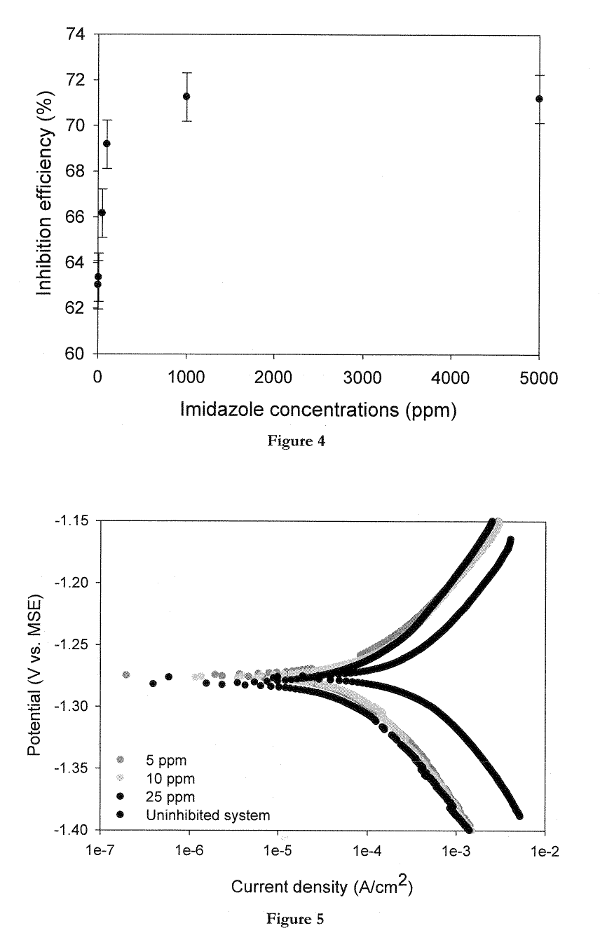

The corrosion rate was calculated based on equation (7) and presented as a function of imidazole concentration as shown in FIG. 3. The corrosion rate decreases in the range of 211-61 mpy as the imidazole concentration is increased (0-1,000 ppm). Upon adding imidazole above 1,000 ppm to 5,000 ppm, there was no significant change of corrosion rate, as it still gives an average corrosion rate of 61 mpy. The inhibition efficiencies of A were calculated by using equation (8), and by comparing the corrosion rates at different concentrations of A with the uninhibited system, yielded inhibition efficiencies in the range of 63-71% as shown in FIG. 4. The optimum concentration of imidazole was 1,000 ppm and the inhibition efficiency was 71%.

While not wishing to be bound by theory it is believed that as imidazole is introduced into the system, it generally adsorbs over the metal surface forming a layer that functions as a barrier protecting the metal from the corrosion as shown in reaction (1). It is thought that imidazole promotes the formation of a chelate on a metal surface, by transferring electrons from its compound to the metal and forming a bond. In this way, the metal acts as an electrophile; and the nucleophile centers of imidazole molecule are normally the lone-pair of electrons on the N-1 atom, through the π-system of the imidazole ring, or through the unshared pair of electrons on the N-3 that are readily available for sharing, to form a bond. This phenomenon may be what is observed from the deviation of the current density at the anodic polarization curve as shown in FIG. 2. The additional imidazole may also slightly reduce the current density at the cathodic side by suppressing the reduction of active agents shown in reaction (2) to (6) due to the adsorbed film on metal surface. The polarization curve shows that the inhibitor can impede both anodic and cathodic processes, but the anodic effect is more pronounced. Adsorption of imidazole on the metal surface reaches a maximum coverage of 71%, which could be confirmed by minimum corrosion rate in FIG. 3 and maximum inhibition efficiency in FIG. 4.

3.1.2 Dodecylamine

Dodecylamine was tested at concentrations of 0, 5, 10, and 25 ppm. Due to the limitation of dodecylamine solubility, higher concentrations of dodecylamine were not carried out. FIG. 5 shows that as dodecylamine concentration increases the current densities of the inhibited system decreases. The shifted polarization curve to lower current density due to the addition of the dodecylamine correlates to lower corrosion rates.

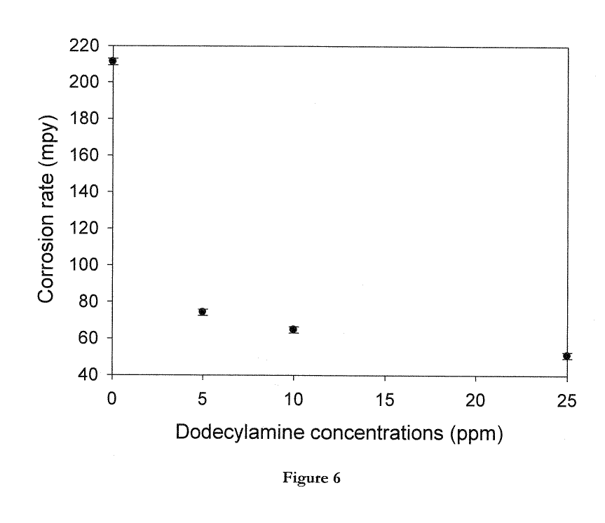

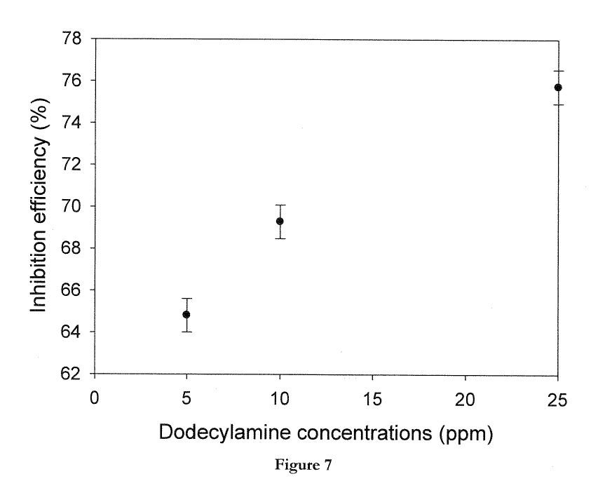

The Tafel plot technique produces anodic and cathodic polarization curves. The corrosion current i_{CORR} can be obtained by the intersection of extrapolating the linear portion of the curve to E_{CORR}. The i_{CORR} can be use to calculate corrosion rate by using equation (7). Generally, a higher i_{CORR} leads to a higher corrosion rate. From the Tafel plots of dodecylamine, the corrosion rate can be calculated from i_{CORR} obtained from the intersection of the linear portions of the polarization curve in both anode and cathode sites at E_{CORR}. FIG. 6 shows the corrosion rates decrease 211, 74, 65, and 51 mpy as the dodecylamine concentrations increase 0, 5, 10, and 25 ppm, respectively. Comparing the corrosion rates of the systems with and without inhibitors, the inhibition efficiency of dodecylamine was found to be 65, 69, and 76% as shown in FIG. 7. It can be concluded that dodecylamine at a concentration of 25 ppm yielded maximum inhibition efficiency of 76%.

While not wishing to be bound by theory it is believed that the corrosion inhibition shown by dodecylamine is due to adsorption of the substance on the metal surface via negatively charged centers on N atom. The hydrophobic part is believed to orientate toward the solution phase reducing access of the corrosive species to the metal surface. Suppression of the oxidation of iron at the anodic sites (reaction (1)) and the reduction of active agents at the cathodic site (reaction (2)-(6)) is believed to occur. This phenomenon may be observed by the deviation of the current density at both the anodic and cathodic polarization curve as shown in FIG. 5. Dodecylamine has a relatively high molecular weight and electron density that tends to establish strong bonds with the metal leading to high inhibition efficiencies even at low concentrations. It is also possible that the large hydrophobic molecular segments, the tails, of the adsorbed molecules extend into the bulk of the solution forming a relatively dense, self-arrangement of projecting tails that remain head-anchored onto the metal surface, and are capable of constituting a corrosive species-repellent layer that affords protection via retarding the mass transfer of the corrosive species to the metal-solution interface.

3.1.3 Sodium Molybdate

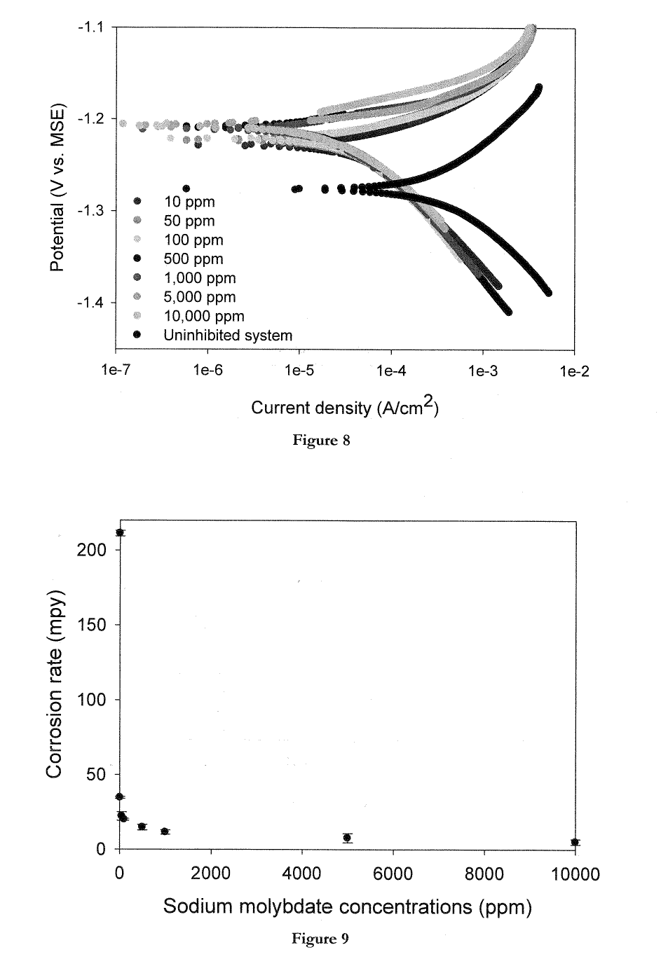

The concentrations of sodium molybdate were varied from 10 to 10,000 ppm. FIG. 8 shows that increasing sodium molybdate concentration causes a significant decrease in current densities. It reveals that the presence of sodium molybdate in the system causes a significant decrease of the current densities at both the anodic and cathodic polarization curve. As mentioned earlier, the shift of polarization curve to the lower current density shows the lower corrosion rate.

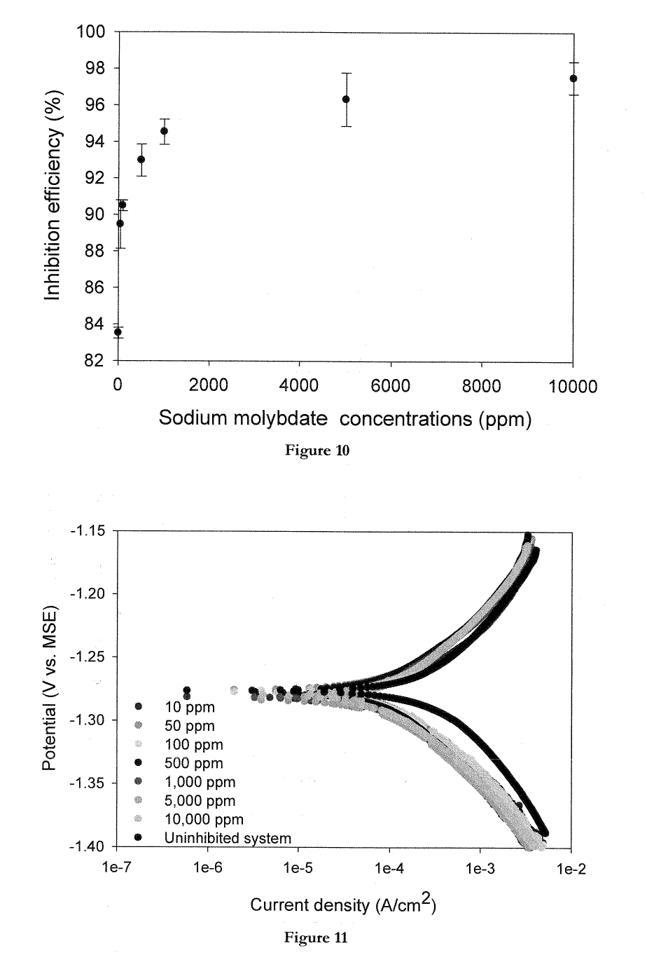

FIG. 9 shows the effect of sodium molybdate concentrations on corrosion rate of carbon steel. It is clearly seen that the corrosion rates decreases tremendously from 211 to 5 mpy as sodium molybdate concentrations increase from 0 to 10,000 ppm. The inhibition efficiency of sodium molybdate was calculated and found to be 84-98% in the range of sodium molybdate concentration of 10-10,000 ppm. Apparently, at 1000 ppm of sodium molybdate, its efficiency was 95%. At the higher sodium molybdate concentration of 5,000 and 10,000 ppm, the inhibition efficiency slightly increased to 96% and 98%, respectively as illustrated in FIG. 10. Due to the cost of the chemical, 1,000 ppm was considered to be an optimum concentration.

While not wishing to be bound by theory the inhibitive action of sodium molybdate could be explained on the basis that the inhibitor strongly adsorbed onto the metal surface. It is thought to form a highly insoluble film of sodium molybdate ions with iron ions which prevents the penetration of corrosive species, thereby decreasing the rate of corrosion.

Fe²⁺+MoO₄²⁻ [symbol] FeMoO₄ (9)

Moreover, when the concentration of inhibitor is high, the surplus sodium molybdate is thought to play a role in the inhibitive efficiency. Sodium molybdate can also react with H⁺ to form the complex ion of sodium molybdate. This ion may react with the steel to form a complex molecule that strongly adsorbs on the surface to further inhibit the corrosion.

[MoO₄²⁻]+H⁺→[MoO₃(OH)]⁻ (10)

The film which is formed between sodium molybdate ion and iron may also suppress the reduction of active agents; it reduces the current density at the cathodic site as shown in FIG. 8. The polarization curve shows that the inhibitor can hinder both anodic and cathodic processes, but the anodic effect seems to be more significant. It is believed that when the film formed by sodium molybdate with iron reaches the maximum coverage, further addition of sodium molybdate into the system does not produce much more inhibition.

3.1.4 Morpholine

The tested morpholine was spiked into the uninhibited system at concentrations of from 0 to 10,000 ppm. FIG. 11 demonstrates that increasing morpholine concentrations shifted the polarization curve to lower current densities. These shifted curves show a lower i_{CORR} which demonstrate lower corrosion rates due to the addition of morpholine.

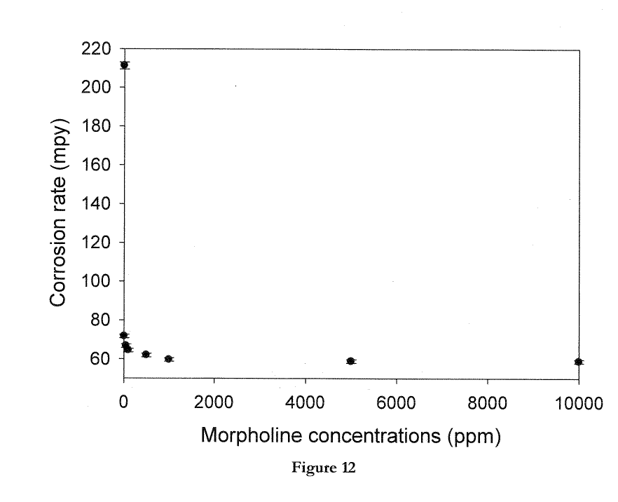

FIG. 12 shows the corrosion rate decreases from 211 to 60 mpy with increasing morpholine concentrations from 0 to 1,000 ppm, and there is no significant change of corrosion rate above 1,000 ppm. At 5,000 and 10,000 ppm the corrosion rate decreased to 59 mpy.

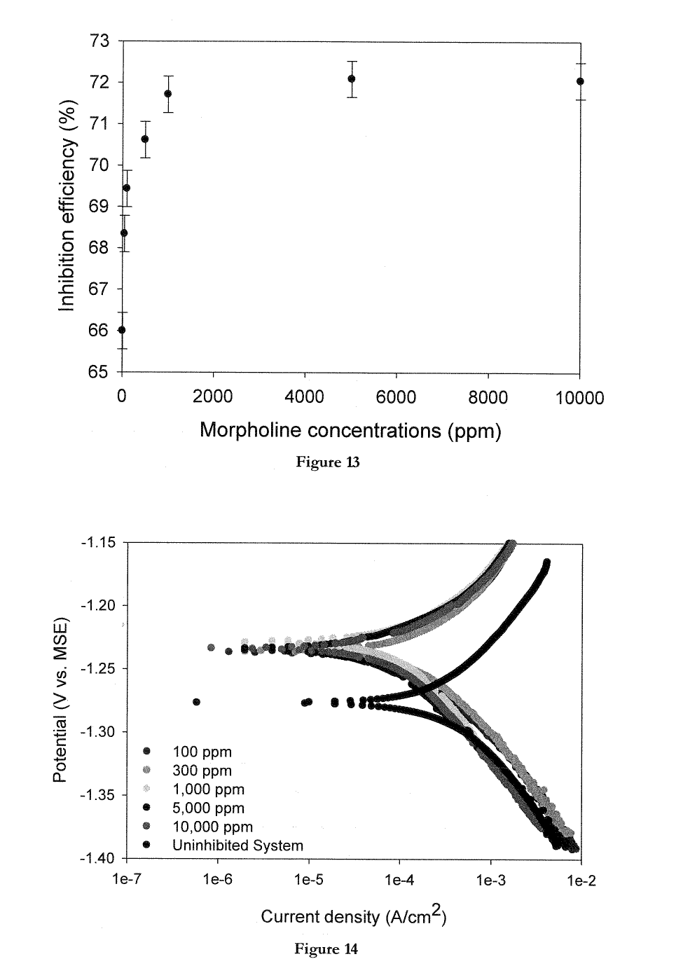

The inhibition efficiency of morpholine was calculated to be 66-72% in the concentration range of 10-1,000 ppm as shown in FIG. 13. The maximum inhibition efficiency was 72% at the optimum concentration of morpholine of 1,000 ppm.

As shown in FIG. 11, the addition of morpholine results in lowering the current densities at the cathodic side. It is believed that this is due to the morpholine taking up H⁺ ions in the solution, which H⁺ is a reducible ion shown in reaction (3). In a MEA-H₂O—CO₂—O₂—SO₂ system, H⁺ can be generated from the following reactions:

SO₂+H₂O [symbol] H⁺+HSO₃⁻ (11)

HSO₃⁻ [symbol] H⁺+SO₃²⁻ (12)

SO₂+½O₂+H₂O [symbol] 2H⁺+SO₄²⁻ (13)

CO₂+H₂O [symbol] H⁺+HCO₃⁻ (14)

HCO₃⁻ [symbol] H⁺+CO₃²⁻ (15)

RRNH+H⁺→RRNH₂⁺ (16)

Morpholine is thought to control the amount of H⁺ generated by reaction (14). The elimination of H⁺ ion may also slightly reduce the oxidation of iron shown in reaction (1), which can be observed by a small reduction of the current density at the anode side as shown in FIG. 11. This polarization curve shows that the inhibitor impedes both anodic and cathodic processes, but the cathodic effect seem to be more significant. When all H⁺ in the system is removed, further addition of morpholine does not appear to further reduce corrosion rate nor improve inhibition efficiency. When the concentration of morpholine is above 1,000 ppm, there is no significant change in both corrosion rate and inhibition efficiency (FIGS. 12 and 13).

3.1.5 Commercial Inhibitor

FIG. 14 shows the polarization curves as varying commercial inhibitor concentrations in 7 kmol/m³ MEA, CO₂ loading of 0.5 mol CO₂/mol MEA solution with simulated flue gas stream of 204 ppm SO₂ and 6% O₂ at 353 K. Commercial inhibitor concentrations were varied between 0 and 10,000 ppm.

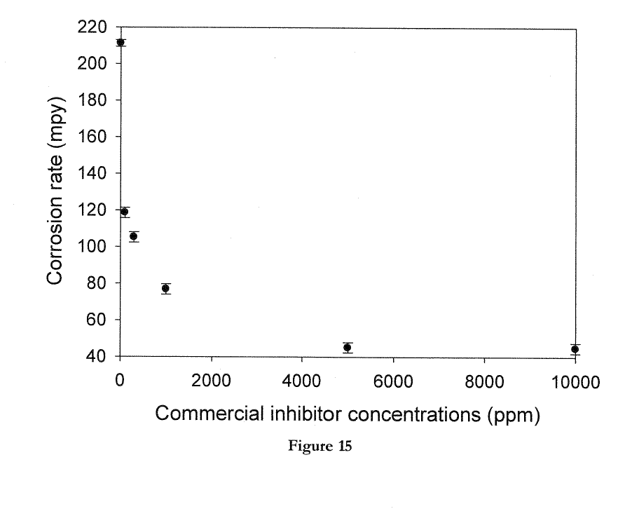

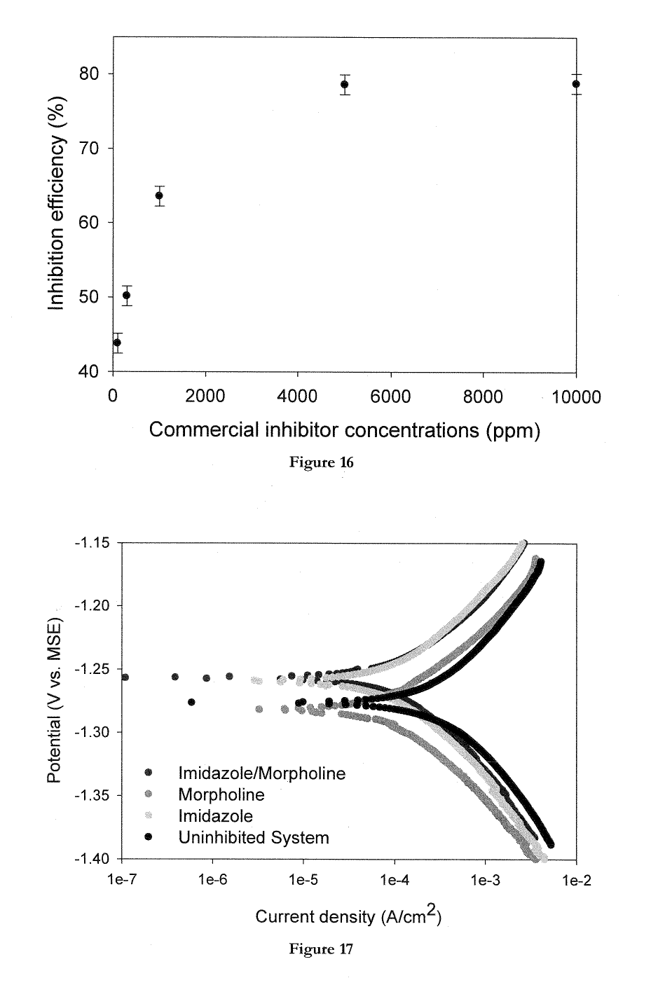

FIG. 15 shows the corrosion rate decreases from 211 to 45 with increasing commercial inhibitor concentrations from 0 to 5,000 ppm, it shows that there was no significant change of corrosion rate above 5,000 ppm. At 10,000 ppm, the corrosion rate of this system was 45 mpy. Commercial inhibitor gave a range of inhibition efficiency varying from 44 to 79% at concentrations from 10 to 1,000 ppm (FIG. 16). 5,000 ppm was considered as an optimum concentration that produced the inhibition efficiency as high as 79%.

Since this inhibitor is a commercial product, its chemical formula and structure are not known. Its mechanism is proposed based on the obtained Tafel plots from the experiment only. The polarization curve shows that commercial inhibitor seems to have a more significant suppression affect on anodic than cathodic sites. The commercial inhibitor appears to suppress the anode site or reaction (1) as compared the inhibited systems have a lower anodic current density than to the uninhibited system as shown in FIG. 14.

3.2. Corrosion System with Blended Inhibitors

Combinations of inhibitors were also tested using morpholine (1,000 ppm) mixed with imidazole (1,000 ppm), dodecylamine (25 ppm), or sodium molybdate (1,000 ppm).

3.2.1 Imidazole/Morpholine

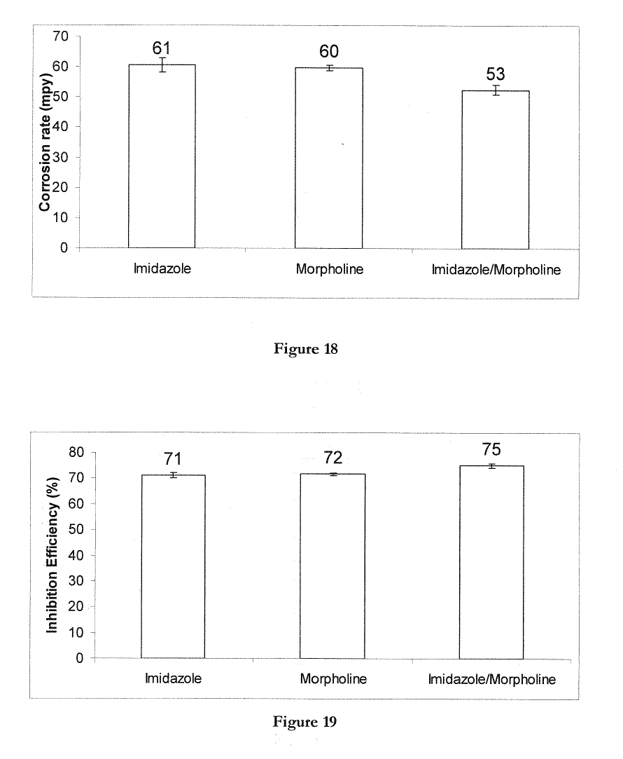

FIG. 17 shows the polarization curves of imidazole, morpholine, and imidazole/morpholine in 7 kmol/m³ MEA, CO₂ loading of 0.5 mol CO₂/mol MEA solution with simulated flue gas stream of 204 ppm SO₂ and 6% O₂ at 353 K. The imidazole/morpholine shows slight effect on lowering current densities than the individual inhibitor. This leads to a slight decrease in corrosion rate as shown in FIG. 18. The imidazole/morpholine can decrease the corrosion rate of carbon steel to 52 mpy corresponding to 75% inhibition efficiency, which is slightly more effective than the single imidazole or morpholine alone as illustrated in FIG. 19.

3.2.2 Dodecylamine/Morpholine

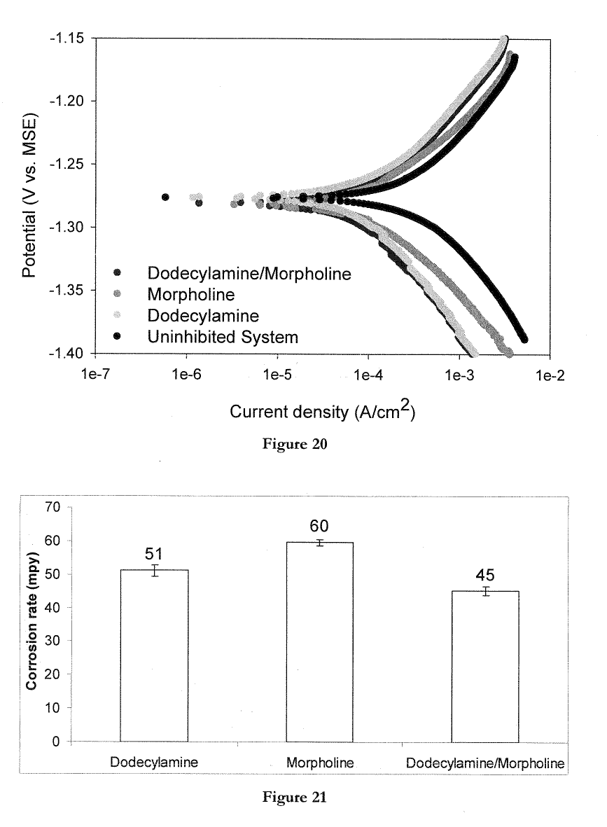

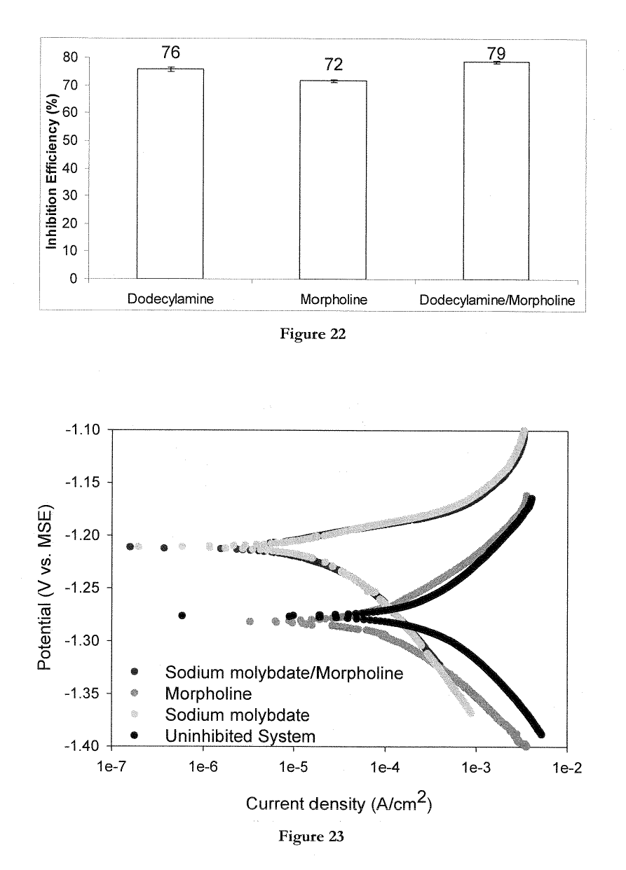

FIG. 20 shows the polarization curves of the addition of dodecylamine, morpholine and dodecylamine/morpholine into the studied system. In FIG. 21 the corrosion rate can be minimized by adding dodecylamine/morpholine to the system. This blended inhibitor yielded the corrosion rate to 45 mpy and 79% of inhibitor efficiency, which is more effective than dodecylamine (76%) or morpholine (72%) alone (FIG. 22).

3.2.3 Sodium Molybdate/Morpholine

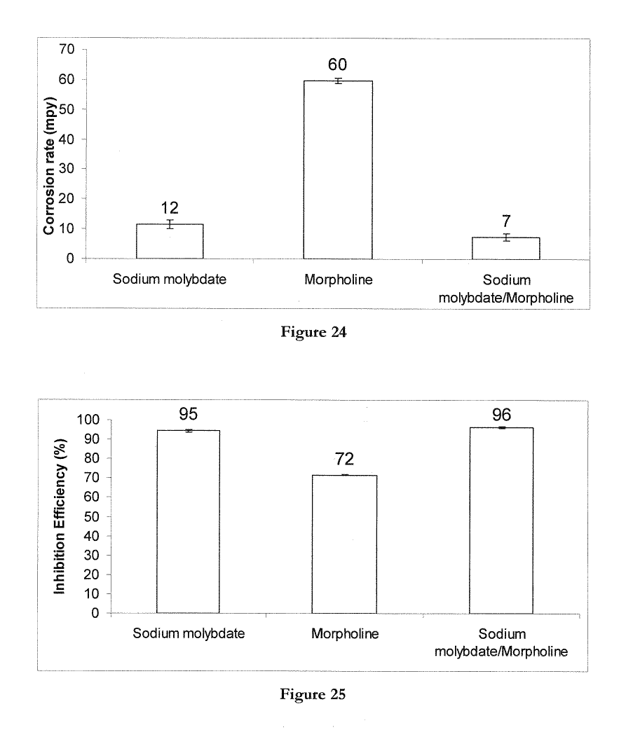

FIG. 23 shows the polarization curve of the effect of sodium molybdate, morpholine, and sodium molybdate/morpholine on corrosion rate of carbon steel in 7 kmol/m³ MEA, CO₂ loading of 0.5 mol CO₂/mol MEA solution with simulated flue gas stream of 204 ppm SO₂ and 6% O₂ at 353 K. FIGS. 24 and 25 show that a blend of sodium molybdate/morpholine yielded the lowest corrosion rate giving 96% inhibition efficiency, whereas sodium molybdate and morpholine individually give inhibition efficiencies of 95% and 72%, respectively. It is shown that sodium molybdate/morpholine could minimize corrosion rate by decreasing its rate to 7 mpy.

Based on the experimental results, dodecylamine, sodium molybdate, morpholine, can reduce corrosion. Blends of morpholine with dodecylamine, sodium molybdate, and imidazole can also reduce corrosion. Inhibition efficiency of each inhibitor also depends on its concentrations which can be concluded as follows.
Dodecylamine—25 ppm; sodium molybdate—1000 ppm; morpholine—1000 ppm; imidazole—1000 ppm; commercial inhibitor—5000 ppm gives inhibition efficiencies of 76%, 95%, 72%, 71%, and 79%, respectively.
Dodecylamine and morpholine are organic inhibitors. Sodium molybdate is inorganic inhibitor.
Blends of imidazole/morpholine, dodecylamine/morpholine, and sodium molybdate/morpholine at individual optimum concentrations were found to enhance the inhibitive effect than their individual compounds.

### REFERENCES

1. Veawab, A.; Tontiwachwuthikul, P.; Chakma, A., Investigation of Low-Toxic Organic Corrosion Inhibitors for CO₂ Separation Process Using Aqueous MEA Solvent. Ind. Eng. Chem. Res. 2001, 40, 4771-4777.
2. Bello, A.; Idem, R. O., Comprehensive Study of the Kinetics of the Oxidative Degradation of CO₂ Loaded and Concentrated Aqueous Monoethanolamine (MEA) with and without Sodium Metavanadate during CO₂ Absorption from Flue Gases. Ind. Eng. Chem. Res. 2006, 45, (8), 2569-2579.
3. Din, A. M. S. E.; Wang, L., Mechanism of corrosion inhibition by sodium molybdate. Desalination 1996, 107, 29-43.
4. Haddad, F.; Benchettara, A.; Amara, S. E.; Kesri, R., Cobalt and Molybdate Ions Effects on the Passivation of Iron Based FeCoC Ternary Alloys, in non Deaerated Solution of 10⁻³ M NaHCO₃+10⁻³ M Na₂SO₄, at 25° C. Portugaliae Electrochimica Acta 2008, 26, 181-198.
5. Zhang, G.; Chena, C.; Lu, M.; Chai, C.; Wu, Y., Evaluation of inhibition efficiency of an imidazoline derivative in CO₂-containing aqueous solution. Materials Chemistry and Physics 2007, 105, 331-340.
6. Zhaoling, L.; Chaoyang, F.; Xingpeng, G., Inhibition performance and adsorption behaviour of dodecylamine on N80 steel in CO₂-saturated brine solution. Anti-Corrosion Methods and Materials 2007, 54, 301-307.
7. Moiseeva, L. S., Finding an Effective Way to Inhibit Carbon Dioxide Corrosion of Ferrous Metals in Oil-and-Gas Production Media. Russian Journal of Applied Chemistry 2004, 77, (8), 1292-1300.
8. Ahmad, Z., Principles of Corrosion Engineering and Corrosion Control. Elsevier Science & Technology Books: 2006.
9. Kladkaew, N.; Idem, R.; Tontiwachwuthikul, P.; Saiwan, C., Corrosion Behavior of Carbon Steel in MEA-H₂O—CO₂—O₂—SO₂ system: Experimental Studies. Ind. Eng. Chem. Res. 2009.
10. Kladkaew, N.; Idem, R.; Tontiwachwuthikul, P.; Saiwan, C., Corrosion Behavior of Carbon Steel in MEA-H₂O—CO₂—O₂—SO₂ system: Products, Reaction Pathways and Kinetics. Ind, Eng. Chem. Res. 2009.
11. Fontana, M. G., Corrosion Engineering. 3rd ed.; McGraw-Hill, Inc.: New York, 1986.
12. Dupart, M. S.; Bacon, T. R.; Edwards, D. J., Part2-Understanding Corrosion in Alkanolamine Gas Treating Plants. Hydrocarbon Processing 1993, (May), 89-94.
13. Horeitz, W., Association of Official Analytical Chemists (AOAC) Methods. 12th ed.; George Banta: 1975.
14. ASTM Standard G1-90 (Reapproved 1999). Standard Practice for Preparing, Cleaning and Evaluating Corrosion Test Specimens. In Annu. Book ASTM Stand., 1999; Vol. 03.01.
15. ASTM Standard G5-94 (Reapproved 2004). Standard Reference Test Method for Making Potentiostatic and Potentiodynamic Anodic Polarization Measurements. In Annul Book ASTM Stand., 2004.
16. Kohl, A. L.; Riesenfeld, F. C., Gas Purification. 3rd ed.; Gulf Publishing Co: Houston, Tex., 1979.
17. POURBAIX, A.; KISSEL, J.; LIGIER, V. ON THE OXIDIZING EFFECT OF SULFITE AND ITS CONSEQUENCES IN MIXED STEEL AND COPPER HEAT EXCHANGE CIRCUITS; Belgian Centre for Corrosion Study: Belgium, 2001; pp 1-11
18. Roberge, P. R., Handbook of Corrosion Engineering. McGraw-Hill: New York, 2000.
19. Zhang, Z.; Chen, S.; Li, Y.; Li, S.; Wang, L., A study of the inhibition of iron corrosion by imidazole and its derivatives self-assembled films. Corrosion Science 2009, 51, 291-300.
20. BereKet, G.; Hur, E.; Ogretir, C., Quantum chemical studies on some imidazole derivatives as corrosion inhibitors for iron in acidic medium. Journal of molecular structure (Theochem) 2002, 578, 79-88.
21. Morales-Gil, P.; Negro'n-Silva, G.; Romero-Romo, M.; ngeles-Cha'vez, C. A.; Palomar-Pardave, M., Corrosion inhibition of pipeline steel grade API 5L X52 immersed in a 1M H2SO4 aqueous solution using heterocyclic organic molecules. Electrochimica Acta 2004, 49, 4733-4741.
22. Abdallah, M.; El-Etre, A. Y.; Soliman, M. G.; Mabrouk, E. M., Some organic and inorganic compounds as inhibitors for carbon steel corrosion in 3.5 percent NaCl solution. Anti-Corrosion Methods and Materials 2006, 53, (2), 118-123.
23. Saremi, M.; Dehghanian, C.; Sabet, M. M., The effect of molybdate concentration and hydrodynamic effect on mild steel corrosion inhibition in simulated cooling water. Corrosion Science 2006, 48, 1404-1412.
24. Mu, G.; Li, X.; Qu, Q.; Zhou, J., Molybdate and tungstate as corrosion inhibitors for cold rolling steel in hydrochloric acid solution. Corrosion Science 2006, 48, 445-459.

1. A process for separating at least a portion of an acid gas from a gaseous mixture, said process comprising:
a. contacting the gaseous mixture with an absorption medium and/or adsorption medium, wherein said medium absorbs and/or adsorbs at least a portion of the acid gas to form an acid gas rich medium;
b. separating at least a portion of the acid gas from the rich medium to form a lean medium;
wherein the absorption medium and/or adsorption medium comprises morpholine.

2. A process for separating at least a portion of an acid gas from a gaseous mixture, said process comprising:
a. contacting the gaseous mixture with an absorption medium and/or adsorption medium, wherein said medium absorbs and/or adsorbs at least a portion of the acid gas to form an acid gas rich medium;
b. separating at least a portion of the acid gas from the rich medium to form a lean medium;
wherein the absorption medium and/or adsorption medium comprises dodecylamine.

3. A process for separating at least a portion of an acid gas from a gaseous mixture, said process comprising:
a. contacting the gaseous mixture with an absorption medium and/or adsorption medium, wherein said medium absorbs and/or adsorbs at least a portion of the acid gas to form an acid gas rich medium;
b. separating at least a portion of the acid gas from the rich medium to form a lean medium;
wherein the absorption medium and/or adsorption medium comprises sodium molybdate.

4. A process according to any preceding claim wherein the acid gas is carbon dioxide.

5. A process according to any preceding claim wherein the absorption/adsorption medium is selected from amines, ammonia, or derivatives/combinations thereof.

6. A process according to any preceding claim wherein the medium is selected from monoethanolamine (MEA), diglycolamine (DGA), diethanolamine (DEA), methyldiethanolamine (MDEA), 2-amino-2-methyl-1-propanol (AMP), piperazine (PZ), ammonia, amines, alkanolamines, derivatives and/or combinations thereof.

7. A process according to any preceding claim wherein the medium is monoethanolamine.

8. Use of a corrosion inhibitor in a process for separating at least a portion of an acid gas from a gaseous mixture wherein the inhibitor is selected from dodecylamine, sodium molybdate, morpholine, or a combination thereof.

9. Use of morpholine for inhibiting corrosion in an acid-gas separation system.

10. Use of a blend of morpholine and dodecylamine in an acid-gas separation system.

11. Use of a blend of morpholine and sodium molybdate in an acid-gas separation system.

12. Use of a blend of morpholine and imidazole in an acid-gas separation system.

13. A use according to any preceding claim wherein the acid gas is carbon dioxide.

14. A use according to any preceding claim wherein the absorption/adsorption medium is selected from amines, ammonia, or derivatives/combinations thereof.

15. A use according to any preceding claim wherein the medium is selected from monoethanolamine (MEA), diglycolamine (DGA), diethanolamine (DEA), methyldiethanolamine (MDEA), 2-amino-2-methyl-1-propanol (AMP), piperazine (PZ), ammonia, amines, alkanolamines, derivatives and/or combinations thereof.

16. A use according to any preceding claim wherein the medium is monoethanolamine.

17. A composition comprising morpholine and dodecylamine.

18. A composition comprising morpholine and sodium molybdate.

19. A composition comprising morpholine and imidazole.
